(19) United States
(12) Patent Application Publication
(10) Pub. No.: US 2004/0002514 A1
(43) Pub. Date: Jan. 1, 2004

(54) Topical use of hydroxyeicosatetraenoic acid compounds to treat psoriasis

(75) Inventor: Daniel A. Gamache, Arlington, TX (US)

Correspondence Address: Alcon Research, Ltd., Patrick M. Ryan, R&D Counsel Q-148, 6201 So. Freeway, Fort Worth, TX, 76134-2099, US

(73) Assignee: Alcon, Inc.

(21) Appl. No.: 10/409,787

(22) Filed: Apr. 9, 2003

Related U.S. Application Data
(60) Provisional application No. 60/389,127, filed on Jun. 14, 2002.

(51) Int. Cl. A61K 31/445; A61K 31/40; A61K 31/23; A61K 31/202; A61K 31/16

(52) U.S. Cl. 514/315; 514/408; 514/560; 514/552; 514/627

(57) ABSTRACT

The topical use of HETE compounds to treat psoriasis is disclosed.

This application claims priority from U.S. Provisional Application, U.S. Serial No. 60/389,127, filed Jun. 14, 2002.

The present invention is directed to the use of hydroxyeicosatetraenoic acid compounds to treat psoriasis. In particular, the invention relates to the topical use of such analogs.

### BACKGROUND OF THE INVENTION

15-Hydroxyeicosatetraenoic acid (“15-HETE”) is known to have inhibitory effects on leukotriene B4 production or its activity. See, for example, Zhu, et al., Skin Pharmacology and Applied Skin Physiology, 13(5):235-45 (September-October 2000); and Heitmann, et al., Experimental Dermatology, 4(2):74-8 (April 1995). 15-HETE is also reported to have minor anti-inflammatory properties in colitis. See Van Dijk, et al., Agents and Actions, 38 Spec. No. C120-1 (1993).

U.S. Pat. No. 5,696,166 (Yanni et al.) discloses compositions containing hydroxyeicosatetraenoic acid (“HETE”) derivatives and methods of using them topically for treating dry eye. Yanni et al. discovered that compositions comprising HETE derivatives increase ocular mucin secretion and are thus useful in treating dry eye.

Reports of the effects of 15-HETE and analogs of 15-HETE on cytokine inhibition in other tissues are varied. See, for example, Denizot, et al., Cytokine, 11(8):606-10 (August 1999) (“ . . . 15-HETE (1 μM to 0.1 nM) [has] no effect on the spontaneous and serum-induced production of IL-8 by human bone marrow stromal cells”); Denizot, et al., Cytokine, 10(10):781-5 (October 1998) (“ . . . 15-HETE . . . [has] no effect on the spontaneous, serum- and cytokine-induced IL-6 synthesis by bone marrow stromal cells; and WO 96/11908, which discloses that certain modified polyunsaturated fatty acids have the ability to suppress cytokine production and cytokine action and are useful as anti-malarial, anti-infective or anti-inflammatory agents.

It has been reported that 15-HETE can improve psoriasis when injected into psoriatic skin lesions. Fogh, et al., “Improvement of Psoriasis Vulgaris After Intralesional Injections of 15-Hydroxyeicosatetraenoic Acid (15-HETE).” J Am Acad Dermatol, 18:279-85 (1988). At page 284 of this article, Fogh, et al. conclude that, in their experiments, 0.1 ml of 10 lmol/L (300 ng) of 15-HETE was required to cause a clinical effect.

### SUMMARY OF THE INVENTION

The present invention is directed to methods of using certain HETE compounds to treat psoriasis. In particular, the present invention is directed toward the topical use of such HETE compounds to treat psoriasis.

### DETAILED DESCRIPTION OF THE INVENTION

As used herein, “HETE compound” or “HETE compounds” means a compound of formulas I-XI.

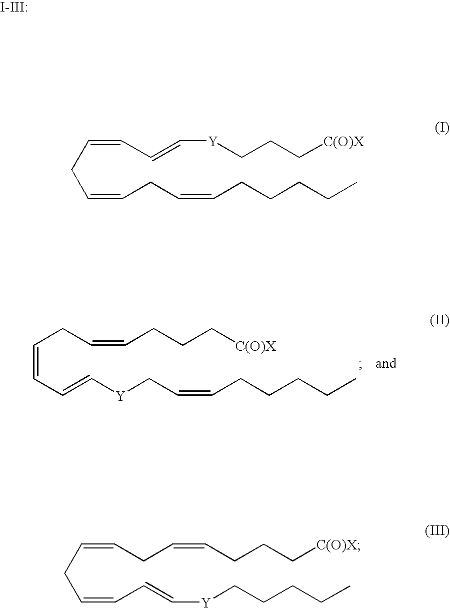

wherein:

X is O⁻M⁺, OR or NHR′;

M⁺0 is Na⁺, K⁺, Li⁺, Cs⁺, and (A)₄N⁺; and A is independently H, alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;

R is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy;

R′ is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy; and

Y is

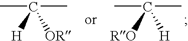

wherein R″ is H or C(O)R;

IV:

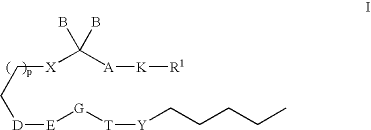

wherein:

R¹ is CO₂R, CONR²R₃, CH₂OR⁴, CH₂NR⁵R₆, CH₂N₃, CH₂-Hal, CH₂NO₂, CH₂SR₂, COSR²⁰, or 2,3,4,5-tetrazol-1-yl, wherein:

R is H or CO₂R forms a pharmaceutically acceptable salt or a pharmaceutically acceptable ester;

NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;

OR⁴ comprises a free or functionally modified hydroxy group, e.g., R⁴ is H, acyl; alkyl, cycloalkyl, aralkyl, or aryl;

Hal is F, Cl, Br or I;

SR²⁰ comprises a free or functionally modified thiol group;

R²¹ is H, or COSR²¹ forms a pharmaceutically acceptable salt or a pharmaceutically acceptable thioester;

K is C₂-C₈ alkyl, alkenyl, or alkynyl, or a C₃-C₈ allenyl group;

A and X are the same or different and are a direct bond, CH₂, NR⁷, O, or S, with the proviso that at least one of A and X is NR⁷, O, or S;

B is H, or BB together comprises a double bonded O, S, or NR⁸, with the proviso that BB comprises a double bonded O, S, or NR when A and X are the same or different and are NR⁷, O, or S; wherein:

NR⁷ and NR⁸ are the same or different and comprise a functionally modified amino group, e.g., R⁷ and R⁸ are the same or different and are H, alkyl, cycloalkyl, aryl, aralkyl, acyl, OH, or alkoxy;

p is 0 or 1;

D-E, G-H are the same or different and are CH₂CH₂, CH═CH, or C≡C; and

Y is C(O) (i.e. a carbonyl group) or Y is

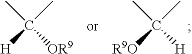

wherein R⁹O constitutes a free or functionally modified hydroxy group; V:

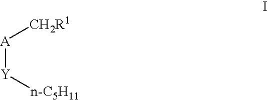

wherein:

R¹ is CO₂R, CONR²R³, CH₂OR₄, CH₂NR⁵R⁶, CH₂N₃, CH₂Hal, CH₂NO₂, CH₂SR²⁰, COSR²¹, or 2,3,4,5-tetrazol-1-yl, where:

R is H or a pharmaceutically acceptable cation, or CO₂R forms a pharmaceutically acceptable ester moiety;

NR²R³, NR⁵R⁶ are the same or different and comprise a free or functionally modified amino group;

OR⁴ comprises a free or functionally modified hydroxy group;

Hal is F, Cl, Br, or I;

R²⁰ is H, alkyl, acyl;

R²¹ is H or a pharmaceutically acceptable cation, or COSR²¹ forms a pharmaceutically acceptable thioester moiety;

A is L₁-A₁-L₂, L₁-A₂-L₂, L₃-A₂-L₄, or L₅-A₂-L₃;

A₁ is CH₂CH₂;

A₂ is

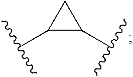

L₁ is CH₂—B-D;

B and D are the same or different and are CH₂CH₂, CH═CH, or C≡C;

L₂ is CH₂—K—CH₂CH₂;

K is CH₂CH₂, CH═CH, or C≡C;

L₃ is CH₂CH₂CH₂, CH₂CH═CH, CH₂C≡C, CH≡CHCH₂, C≡CCH₂, or CH═C═CH;

L₄ is X—CH₂CH₂;

X is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C═CHCH₂;

L₅ is CH₂CH₂—B-D; and

Y is C(O) (i.e. a carbonyl group) or Y is

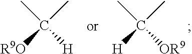

wherein R⁹O constitutes a free or functionally modified hydroxy group;

VI:

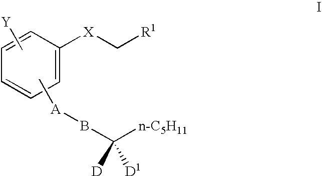

wherein:

R¹ is CO₂R, CONR²R₃, CH₂OR⁴, CH₂NR⁵R⁶, CH₂N₃, CH₂Hal, CH₂NO₂, CH₂SR²⁰, COSR²¹ or 2,3,4,5 tetrazol-1-yl, wherein:

R is H or CO₂R forms a pharmaceutically acceptable salt or a pharmaceutically acceptable ester;

NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH or alkoxy, with the proviso that at most only one of R² and R³ are OH or alkoxy and at most only one of R and R are OH or alkoxy;

OR⁴ comprises a free or functionally modified hydroxy group, e.g., R⁴ is H, acyl; alkyl, cycloalkyl, aralkyl or aryl;

Hal is F, Cl, Br or I;

SR²⁰ comprises a free or functionally modified thiol group;

R²¹ is H or COSR²¹ forms a pharmaceutically acceptable salt or a pharmaceutically acceptable thioester;

X is C₂-C₅ alkyl, alkynyl, or alkenyl or a C₃-C₅ allenyl group;

Y is H, free or functionally modified hydroxy group, halo, trihalomethyl, free or functionally modified amino group, free or functionally modified thiol group, C(O)R⁷, or alkyl;

R⁷ is H, OH, alkyl, alkoxy, amino, alkylamino or alkoxyamino;

A is a direct bond or C₁₋₃ alkyl;

B is CH₂CH₂, cis- or trans-CH═CH, or C≡C; and

one of D and D¹ is H and the other is a free or functionally modified OH group, or DD¹ together comprises a double bonded oxygen;

VII:

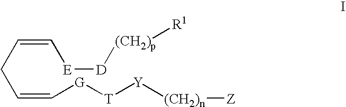

wherein:

R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:

R is H or CO₂R forms a pharmaceutically acceptable salt or a pharmaceutically acceptable ester;

NR²R³ and NR⁵R are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;

OR⁴ comprises a free or functionally modified hydroxy group, e.g., R⁴ is H, acyl; alkyl, cycloalkyl, aralkyl or aryl;

Hal is F, Cl, Br or I;

SR²⁰ comprises a free or functionally modified thiol group;

R²¹ is H or COSR²¹ forms a pharmaceutically acceptable salt or a pharmaceutically acceptable thioester;

E-D is CH₂CH₂CH₂ or cis-CH₂CH═CH; or E is trans-CH═CH and D is CH(OH) in either configuration, wherein the OH is free or functionally modified; or E is CH₂CH₂ and D is a direct bond;

p is 1 or 3 when E-D is CH₂CH₂CH₂ or cis-CH₂CH═CH, or when E is trans-CH═CH and D is CH(OH) in either configuration, wherein the OH is free or functionally modified; or p is 0 when E is CH₂CH₂ and D is a direct bond; G-T is CH₂CH₂, CH(SR⁷)CH₂ or trans-CH═CH;

R⁷ is H, alkyl, aryl, aralkyl, cycloalkyl or acyl;

Y is CH(OH) in either configuration, in which the OH is free of functionally modified, or C═O (i.e., a carbonyl group);

n is 0, 2 or 4; and

Z is CH₃, CO₂R, CONR²R³ or CH₂OR⁴;

VII:

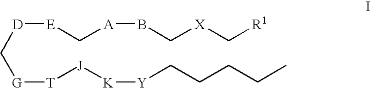

wherein:

R¹ is (CH₂)ₙCO₂R, (CH₂)ₙCONR²R³, (CH₂)ₙCH₂OR⁴, (CH₂)ₙCH₂NR⁵R⁶ (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:

R is H or CO₂R forms a pharmaceutically acceptable salt or a pharmaceutically acceptable ester;

NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;

OR⁴ comprises a free or functionally modified hydroxy group, e.g., R⁴ is H, acyl; alkyl, cycloalkyl, aralkyl, or aryl;

Hal is F, Cl, Br or I;

SR²⁰ comprises a free or functionally modified thiol group;

R²¹ is H or COSR²¹ forms a pharmaceutically acceptable salt or a pharmaceutically acceptable thioester;

n is 0 or 2;

X is O, S(O)ₚ, NR⁷ or CH₂, with the proviso that X cannot be CH₂ when n is 0;

p is 0, 1 or 2;

NR⁷ comprises a free or functionally modified amino group, e.g., R⁷ is H, alkyl, cycloalkyl, aralkyl, aryl, OH or alkoxy,

A-B, D-E, G-T and J-K are the same or different and are CH₂CH₂, CH═CH or C≡C, with the proviso that at least one of A-B, D-E, G-T and J-K must be CH═CH or C≡C; and

Y is C(O) (i.e., a carbonyl), or Y is

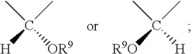

wherein R⁹O constitutes a free or functionally modified hydroxy group;

IX:

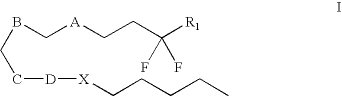

wherein:

R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:

R is H or CO₂R forms a pharmaceutically acceptable salt or a pharmaceutically acceptable ester;

NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;

OR⁴ comprises a free or functionally modified hydroxy group, e.g., R⁴ is H, acyl; alkyl, cycloalkyl, aralkyl, or aryl;

Hal is F, Cl, Br or I;

SR²⁰ comprises a free or functionally modified thiol group;

R²¹ is H or COSR²¹ forms a pharmaceutically acceptable salt or a pharmaceutically acceptable thioester;

A, B, C and D are the same or different and are C₁-C₅ alkyl, alkenyl, or alkynyl or a C₃-C₅ allenyl group;

X is C(O) (i.e. a carbonyl group) or X is

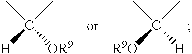

wherein R⁹O constitutes a free or functionally modified hydroxy group;

X:

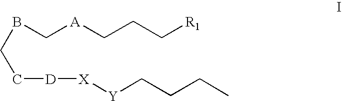

wherein:

R¹ is (CH₂)ₙCO₂R, (CH₂)ₙCONR²R³, (CH₂)ₙCH₂OR⁴, (CH₂)ₙCH₂NR⁵R⁶, (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:

R is H or CO₂R forms a pharmaceutically acceptable salt or a pharmaceutically acceptable ester;

NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;

OR⁴ comprises a free or functionally modified hydroxy group, e.g., R⁴ is H, acyl; alkyl, cycloalkyl, aralkyl, or aryl;

Hal is F, Cl, Br or I;

SR²⁰ comprises a free or functionally modified thiol group;

R²¹ is H or COSR²¹ forms a pharmaceutically acceptable salt or a pharmaceutically acceptable thioester;

n is 0 or 2;

A, B, C and D is C₁-C₅ alkyl, alkenyl, or alkynyl or a C₃-C₅ allenyl group;

Y is

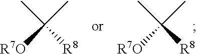

wherein R⁸ is H or CH₃, and

X is CH₂, CH(CH₃) or C(CH₃)₂; or

Y is CH₂, CH(CH₃) or C(CH₃)₂, and X is

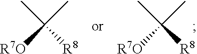

wherein R⁸ is H or CH₃, with the proviso that Y cannot be CH₂ when X is

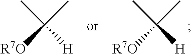

and

R⁷O comprises a free or functionally modified hydroxy group; and

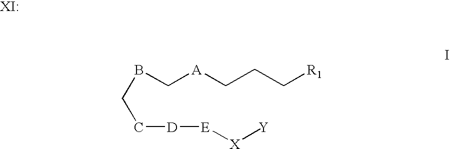

wherein:

R¹ is CO₂R, CONR²R³, CH₂OR⁴, CH₂NR⁵R⁶, CH₂N₃, CH₂Hal, CH₂NO₂, CH₂SR²⁰, COSR²¹, or 2,3,4,5-tetrazol-1-yl, where:

R is H or a pharmaceutically acceptable cation, or CO₂R forms a pharmaceutically acceptable ester moiety;

NR²R³, NR⁵R⁶ are the same or different and comprise a free or functionally modified amino group;

OR⁴ comprises a free or functionally modified hydroxy group;

Hal is F, Cl, Br, or I;

SR²⁰ comprises a free or functionally modified thiol group;

R²¹ is H or a pharmaceutically acceptable cation, or COSR²¹ forms a pharmaceutically acceptable thioester moiety;

A, B, C, D are the same or different and are C₁-C₅ alkyl, C₂-C₅ alkenyl, C₁₋₅ cyclopropyl, C₂-C₅ alkynyl, or a C₃-C₅ allenyl group;

E is

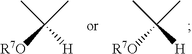

where OR⁷ comprises a free or functionally modified hydroxy group;

X═(CH₂)ₘ or (CH₂)ₘO, where m=1-6; and

Y=a phenyl ring optionally substituted with alkyl, halo, trihalomethyl, acyl, or a free or functionally modified hydroxy, amino, or thiol group; or

X—Y═(CH₂)ₚY¹; where p=0-6; and

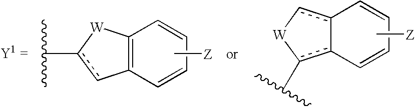

wherein:

W=CH₂, O, S(O)_{q}, NR⁸, CH₂CH₂, CH═CH, CH₂O, CH₂S(O)_{q}, CH═N, or CH₂NR⁸; where q=0-2, and R⁸═H, alkyl, or acyl;

Z=H, alkyl, acyl, halo, trihalomethyl, or a free or functionally modified amino, thiol, or hydroxy group; and

----=single or double bond;

or X—Y=cyclohexyl.

Preferred HETE compounds include the compounds of formulas I-III wherein X is a pharmaceutically acceptable salt containing a cation selected from the group consisting of: Na⁺; K⁺; Li⁺; Cs⁺; and (A)₄N⁺; and A is independently H, alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring.

Included within the scope of the present invention are the individual enantiomers of the HETE compounds, as well as their racemic and non-racemic mixtures. The individual enantiomers can be enantioselectively synthesized from the appropriate enantiomerically pure or enriched starting material by means such as those described below. Alternatively, they may be enantioselectively synthesized from racemic/non-racemic or achiral starting materials. (Asymmetric Synthesis; J. D. Morrison and J. W. Scott, Eds.; Academic Press Publishers: New York, 1983-1985, volumes 1-5; Principles of Asymmetric Synthesis; R. E. Gawley and J. Aube, Eds.; Elsevier Publishers: Amsterdam, 1996). They may also be isolated from racemic and non-racemic mixtures by a number of known methods, e.g. by purification of a sample by chiral HPLC (A Practical Guide to Chiral Separations by HPLC; G. Subramanian, Ed.; VCH Publishers: New York, 1994; Chiral Separations by HPLC; A. M. Krstulovic, Ed.; Ellis Horwood Ltd. Publishers, 1989), or by enantioselective hydrolysis of a carboxylic acid ester sample by an enzyme (Ohno, M.; Otsuka, M. Organic Reactions, volume 37, page 1 (1989)). Those skilled in the art will appreciate that racemic and non-racemic mixtures may be obtained by several means, including without limitation, nonenantioselective synthesis, partial resolution, or even mixing samples having different enantiomeric ratios. Departures may be made from such details within the scope of the accompanying claims without departing from the principles of the invention and without sacrificing its advantages. Also included within the scope of the present invention are the individual isomers substantially free of their respective enantiomers.

The term “free hydroxy group” means an OH. The term “functionally modified hydroxy group” means an OH which has been functionalized to form: an ether, in which an alkyl, aryl, cycloalkyl, heterocycloalkyl, alkenyl, cycloalkenyl, heterocycloalkenyl, alkynyl, or heteroaryl group is substituted for the hydrogen; an ester, in which an acyl group is substituted for the hydrogen; a carbamate, in which an aminocarbonyl group is substituted for the hydrogen; or a carbonate, in which an aryloxy-, heteroaryloxy-, alkoxy-, cycloalkoxy-, heterocycloalkoxy-, alkenyloxy-, cycloalkenyloxy-, heterocycloalkenyloxy-, or alkynyloxy-carbonyl group is substituted for the hydrogen. Preferred moieties include OH, OCH₂C(O)CH₃,OCH₂C(O)C₂H₅, OCH₃, OCH₂CH₃, OC(O)CH₃, and OC(O)C₂H₅.

The term “free amino group” means an NH₂. The term “functionally modified amino group” means an NH₂ which has been functionalized to form: an aryloxy-, heteroaryloxy-, alkoxy-, cycloalkoxy-, heterocycloalkoxy-, alkenyl-, cycloalkenyl-, heterocycloalkenyl-, alkynyl-, or hydroxy-amino group, wherein the appropriate group is substituted for one of the hydrogens; an aryl-, heteroaryl-, alkyl-, cycloalkyl-, heterocycloalkyl-, alkenyl-, cycloalkenyl-, heterocycloalkenyl-, or alkynyl-amino group, wherein the appropriate group is substituted for one or both of the hydrogens; an amide, in which an acyl group is substituted for one of the hydrogens; a carbamate, in which an aryloxy-, heteroaryloxy-, alkoxy-, cycloalkoxy-, heterocycloalkoxy-, alkenyl-, cycloalkenyl-, heterocycloalkenyl-, or alkynyl-carbonyl group is substituted for one of the hydrogens; or a urea, in which an aminocarbonyl group is substituted for one of the hydrogens. Combinations of these substitution patterns, for example an NH₂ in which one of the hydrogens is replaced by an alkyl group and the other hydrogen is replaced by an alkoxycarbonyl group, also fall under the definition of a functionally modified amino group and are included within the scope of the present invention. Preferred moieties include NH₂, NHCH₃, NHC₂H₅, N(CH₃)₂, NHC(O)CH₃, NHOH, and NH(OCH₃).

The term “free thiol group” means an SH. The term “functionally modified thiol group” means an SH which has been functionalized to form: a thioether, where an alkyl, aryl, cycloalkyl, heterocycloalkyl, alkenyl, cycloalkenyl, heterocycloalkenyl, alkynyl, or heteroaryl group is substituted for the hydrogen; or a thioester, in which an acyl group is substituted for the hydrogen. Preferred moieties include SH, SC(O)CH₃, SCH₃, SC₂H₅, SCH₂C(O)C₂H₅, and SCH₂C(O)CH₃.

The term “acyl” represents a group that is linked by a carbon atom that has a double bond to an oxygen atom and a single bond to another carbon atom.

The term “alkyl” includes straight or branched chain aliphatic hydrocarbon groups that are saturated and have 1 to 15 carbon atoms. The alkyl groups may be interrupted by one or more heteroatoms, such as oxygen, nitrogen, or sulfur, and may be substituted with other groups, such as halogen, hydroxyl, aryl, cycloalkyl, aryloxy, or alkoxy. Preferred straight or branched alkyl groups include methyl, ethyl, propyl, isopropyl, butyl and t-butyl.

The term “cycloalkyl” includes straight or branched chain, saturated or unsaturated aliphatic hydrocarbon groups which connect to form one or more rings, which can be fused or isolated. The rings may be substituted with other groups, such as halogen, hydroxyl, aryl, aryloxy, alkoxy, or lower alkyl. Preferred cycloalkyl groups include cyclopropyl, cyclobutyl, cyclopentyl and cyclohexyl.

The term “C₁-C₅ cyclopropyl” means an alkyl chain of 1 to 5 carbon atoms containing a cyclopropyl group wherein the cyclopropyl group may start, be contained in or terminate the alkyl chain.

The term “heterocycloalkyl” refers to cycloalkyl rings that contain at least one heteroatom such as O, S, or N in the ring, and can be fused or isolated. The rings may be substituted with other groups, such as halogen, hydroxyl, aryl, aryloxy, alkoxy, or lower alkyl. Preferred heterocycloalkyl groups include pyrrolidinyl, tetrahydrofuranyl, piperazinyl, and tetrahydropyranyl.

The term “alkenyl” includes straight or branched chain hydrocarbon groups having 1 to 15 carbon atoms with at least one carbon-carbon double bond, the chain being optionally interrupted by one or more heteroatoms. The chain hydrogens may be substituted with other groups, such as halogen. Preferred straight or branched alkenyl groups include, allyl, 1-butenyl, 1-methyl-2-propenyl and 4-pentenyl.

The term “cycloalkenyl” includes straight or branched chain, saturated or unsaturated aliphatic hydrocarbon groups which connect to form one or more non-aromatic rings containing a carbon-carbon double bond, which can be fused or isolated. The rings may be substituted with other groups, such as halogen, hydroxyl, alkoxy, or lower alkyl. Preferred cycloalkenyl groups include cyclopentenyl and cyclohexenyl.

The term “heterocycloalkenyl” refers to cycloalkenyl rings which contain one or more heteroatoms such as O, N, or S in the ring, and can be fused or isolated. The rings may be substituted with other groups, such as halogen, hydroxyl, aryl, aryloxy, alkoxy, or lower alkyl. Preferred heterocycloalkenyl groups include pyrrolidinyl, dihydropyranyl, and dihydrofuranyl.

The term “carbonyl group” represents a carbon atom double bonded to an oxygen atom, wherein the carbon atom has two free valencies.

The term “aminocarbonyl” represents a free or functionally modified amino group bonded from its nitrogen atom to the carbon atom of a carbonyl group, the carbonyl group itself being bonded to another atom through its carbon atom.

The term “lower alkyl” represents alkyl groups containing one to six carbons (C₁-C₆).

The term “halogen” represents fluoro, chloro, bromo, or iodo.

The term “aryl” refers to carbon-based rings which are aromatic. The rings may be isolated, such as phenyl, or fused, such as naphthyl. The ring hydrogens may be substituted with other groups, such as lower alkyl, halogen, free or functionalized hydroxy, trihalomethyl, etc. Preferred aryl groups include phenyl, 3-(trifluoromethyl)phenyl, 3-chlorophenyl, and 4-fluorophenyl.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

The terms “a ryloxy”, “heteroa ryloxy”, “al koxy”, “cycloal koxy”, “heterocycloalkoxy”, “al kenyloxy”, “cycloal kenyloxy”, “heterocycloalkenyloxy”, and “alkynyloxy” represent an aryl, heteroaryl, alkyl, cycloalkyl, heterocycloalkyl, alkenyl, cycloalkenyl, heterocycloalkenyl, or alkynyl group, respectively, attached through an oxygen linkage.

The terms “alkoxycarbonyl”, “aryloxycarbonyl”, “heteroaryloxycarbonyl”, “cycloalkoxycarbonyl”, “heterocycloal koxycarbonyl”, “alkenyloxycarbonyl”, “cycloalkenyloxycarbonyl”, “heterocycloalkenyloxycarbonyl”, and “alkynyloxycarbonyl” represent an alkoxy, aryloxy, heteroaryloxy, cycloalkoxy, heterocycloalkoxy, alkenyloxy, cycloalkenyloxy, heterocycloalkenyloxy, or alkynyloxy group, respectively, bonded from its oxygen atom to the carbon of a carbonyl group, the carbonyl group itself being bonded to another atom through its carbon atom.

According to the methods of the present invention a HETE compound of formulas I-XI is applied topically to the skin at the site of a psoriatic lesion. The compositions used in the methods of the present invention comprise a pharmaceutically effective amount of one or more HETE compounds of formulas I-XI and a pharmaceutically acceptable carrier. Suitable pharmaceutical carriers are known in the art and include, but are not limited to, dermatologically acceptable solutions, suspensions, creams and ointments. Aqueous solutions are generally preferred, based on ease of formulation, biological compatibility, as well as a patient's ability to easily administer such compositions. However, the compositions may also be suspensions, viscous or semi-viscous gels, or other types of solid or semi-solid compositions.

As used herein, the term “pharmaceutically effective amount” refers to an amount of one or more compounds of formulas I-XI that, when administered to a patient, reduces or eliminates psoriasis. Generally, the compounds of formulas I-XI will be contained in a composition of the present invention in a concentration range of about 0.000001 to 10 percent weight/volume (“% w/v”). Preferably, the compositions will contain one or more compounds of formulas I-XI in a concentration of from about 0.00001-0.01% w/v.

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. In general, however, the buffering agent will be present in an amount sufficient to hold the pH within the range 6.5-8.0, preferably 6.8-7.6.

Antioxidants may be added to compositions of the present invention to protect the compounds of formulas I-XI from oxidation during storage. Examples of such antioxidants include, but are not limited to, vitamin E and analogs thereof, ascorbic acid and derivatives, and butylated hydroxyanisole (BHA).

The following examples are presented to illustrate various aspects of the present invention, but are not intended to limit the scope of the invention in any respect.

Example 1

| Ingredient | Amount (% w/v) |
|---|---|
| Compound of formulas I-XI | 0.00001-0.01 |
| Ethanol | 0.0505 |
| Polyoxyl 40 Stearate | 0.1 |
| Boric Acid | 0.25 |
| Sodium Chloride | 0.75 |
| Disodium Edetate | 0.01 |
| Polyquaternium-1 | 0.001 |
| NaOH/HCl | q.s., pH = 7.5 |
| Purified Water | q.s. 100% |

The above composition is prepared by the following method. The batch quantities of polyoxyl 40 stearate, boric acid, sodium chloride, disodium edetate, and polyquaternium-1 are weighed and dissolved by stirring in 90% of the batch quantity of purified water. The pH is adjusted to 7.5±0.1 with NaOH and/or HCl. Under yellow light or reduced lighting, the batch quantity of the compound of formulas I-XI as a stock solution in ethanol and the additional quantity of ethanol necessary for the batch are measured and added. Purified water is added to q.s. to 100%. The mixture is stirred for five minutes to homogenize and then filtered through a sterilizing filter membrane into a sterile recipient. Preferably, the above process is performed using glass, plastic or other non-metallic containers or containers lined with such materials.

Example 2

| Ingredient | Amount (% w/v) |
|---|---|
| Compound of formulas I-XI | 0.00001-0.01 |
| Polyoxyl 40 Stearate | 0.1 |
| Boric Acid | 0.25 |
| Sodium Chloride | 0.75 |
| Disodium Edetate | 0.01 |
| Polyquaternium-1 | 0.001 |
| NaOH/HCl | q.s., pH = 6.5-8 |
| Purified Water | q.s. 100% |

The above formulation may be made by a method similar to the method described in Example 1.

Example 3

| Ingredient | Amount (% w/v) |
|---|---|
| Compound of formulas I-XI | 0.00001-0.01 |
| Polyoxyl 40 Stea rate | 0.1 |
| Ethanol | 0.005-0.2 |
| Boric Acid | 0.25 |
| Sodium Chloride | 0.75 |
| NaOH/HCl | q.s., pH = 6.5-8 |
| Purified Water | q.s. 100% |

The above formulation may be made by a method similar to the method described in Example 1.

The invention in its broader aspects is not limited to the specific details shown and described above. Departures may be made from such details within the scope of the accompanying claims without departing from the principles of the invention and without sacrificing its advantages.

What is claimed is:

1. A method of treating psoriasis in a patient, wherein the method comprises topically administering to the patient a composition comprising a dermatologically acceptable carrier and a pharmaceutically effective amount of a HETE compound of formulas I-XI:
I-III: 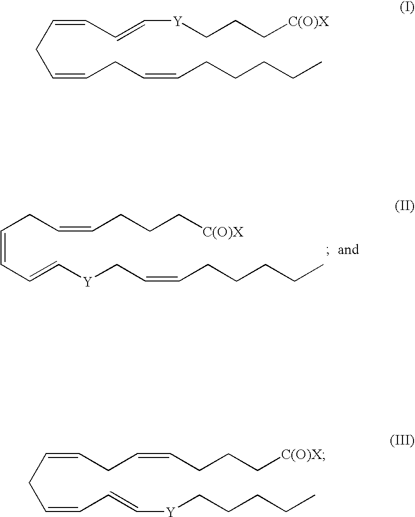
wherein:
X is O⁻M⁺, OR or NHR′;
M⁺ is Na⁺, K⁺, Li⁺, Cs⁺, and (A)₄N⁺; and A is independently H, alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy;
R′ is H, substituted or unsubstituted alkyl, cycloalkyl, (cycloalkyl)alkyl, aryl, arylalkyl, wherein the substitution is made with a moiety selected from the group consisting of: alkyl, halogen, hydroxy and functionally modified hydroxy; and
Y is 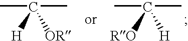
wherein R″ is H or C(O)R;
IV: 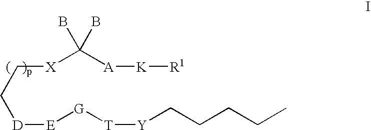
wherein:
R¹ is CO₂R, CONR²R³, CH₂OR⁴, CH₂NR⁵R⁶, CH₂N₃, CH₂-Hal, CH₂NO₂, CH₂SR²⁰, COSR²¹, or 2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl, or aryl;
Hal is F, Cl, Br or I;
SR²⁰ comprises a free or functionally modified thiol group;
R²¹ is H, or COSR²¹ forms a pharmaceutically acceptable salt or a pharmaceutically acceptable thioester;
K is C₂-C₈ alkyl, alkenyl, or alkynyl, or a C₃-C₈ allenyl group;
A and X are the same or different and are a direct bond, CH₂, NR⁷, O, or S, with the proviso that at least one of A and X is NR⁷, O, or S;
B is H, or BB together comprises a double bonded O, S, or NR⁸, with the proviso that BB comprises a double bonded O, S, or NR when A and X are the same or different and are NR⁷, 0, or S; wherein:
NR⁷ and NR⁸ are the same or different and comprise a functionally modified amino group, e.g., R⁷ and R⁸ are the same or different and are H, alkyl, cycloalkyl, aryl, aralkyl, acyl, OH, or alkoxy;
p is 0 or 1;
D-E, G-H are the same or different and are CH₂CH₂, CH═CH, or C≡C; and
Y is C(O) (i.e. a carbonyl group) or Y is 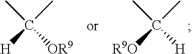
wherein R⁹O constitutes a free or functionally modified hydroxy group;
V: 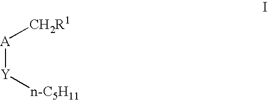
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹, or 2,3,4,5-tetrazol-1-yl, where:
R is H or a pharmaceutically acceptable cation, or CO₂R forms a pharmaceutically acceptable ester moiety;
NR²R³, NR⁵R⁶ are the same or different and comprise a free or functionally modified amino group;
OR⁴ comprises a free or functionally modified hydroxy group;
Hal is F, Cl, Br, or I;
R²⁰ is H, alkyl, acyl;
R²¹ is H or a pharmaceutically acceptable cation, or COSR²¹ forms a pharmaceutically acceptable thioester moiety;
A is L₁-A₁-L₂, L₁-A₂-L₂, L₃-A₂-L₄, or L₅-A₂-L₃;
A₁ is CH₂CH₂;
A₂ is 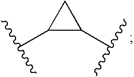
L₁ is CH₂—B-D;
B and D are the same or different and are CH₂CH₂, CH═CH, or C≡C;
L₂ is CH₂—K—CH₂CH₂;
K is CH₂CH₂, CH═CH, or C≡C;
L₃ is CH₂CH₂CH₂, CH₂CH═CH, CH₂C≡C, CH═CHCH₂, C≡CCH₂, or CH═C═CH;
L₄ is X—CH₂CH₂;
X is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C═CHCH₂;
L₅ is CH₂CH₂—B-D; and
Y is C(O) (i.e. a carbonyl group) or Y is 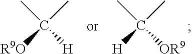
wherein R⁹O constitutes a free or functionally modified hydroxy group;
VI: 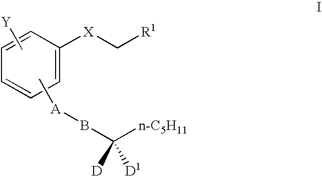
wherein:
R¹ is CO₂R, CONR²R₃, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl or aryl;
Hal is F, Cl, Br or I;
SR²⁰ comprises a free or functionally modified thiol group;
R²¹ is H or COSR²¹ forms a pharmaceutically acceptable salt or a pharmaceutically acceptable thioester;
X is C₂-C₅ alkyl, alkynyl, or alkenyl or a C₃-C₅ allenyl group;
Y is H, free or functionally modified hydroxy group, halo, trihalomethyl, free or functionally modified amino group, free or functionally modified thiol group, C(O)R⁷, or alkyl;
R⁷ is H, OH, alkyl, alkoxy, amino, alkylamino or alkoxyamino;
A is a direct bond or C₁₋₃ alkyl;
B is CH₂CH₂, cis- or trans-CH═CH, or C≡C; and
one of D and D¹ is H and the other is a free or functionally modified OH group, or DD¹ together comprises a double bonded oxygen;
VII: 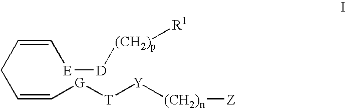
wherein:
R¹ is CO₂R, CONR²R³, CH₂OR₁, CH₂NR⁵R⁶, CH₂N₃, CH₂Hal, CH₂NO₂, CH₂SR²⁰, COSR²¹ or 2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl or aryl;
Hal is F, Cl, Br or I;
SR²⁰ comprises a free or functionally modified thiol group;
R²¹ is H or COSR²¹ forms a pharmaceutically acceptable salt or a pharmaceutically acceptable thioester;
E-D is CH₂CH₂CH₂ or cis-CH₂CH═CH; or E is trans-CH═CH and D is CH(OH) in either configuration, wherein the OH is free or functionally modified; or E is CH₂CH₂ and D is a direct bond;
p is 1 or 3 when E-D is CH₂CH₂CH₂ or cis-CH₂CH═CH, or when E is trans-CH═CH and D is CH(OH) in either configuration, wherein the OH is free or functionally modified; or p is 0 when E is CH₂CH₂ and D is a direct bond;
G-T is CH₂CH₂, CH(SR⁷)CH₂ or trans-CH═CH;
R⁷ is H, alkyl, aryl, aralkyl, cycloalkyl or acyl;
Y is CH(OH) in either configuration, in which the OH is free of functionally modified, or C═O (i.e., a carbonyl group);
n is 0, 2 or 4; and
Z is CH₃, CO₂R, CONR²R³ or CH₂OR⁴;
VIII: 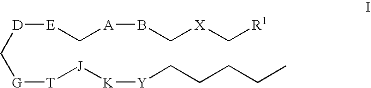
wherein:
R¹ is (CH₂)ₙCO₂R, (CH₂)ₙCONR²R³, (CH₂)ₙCH₂OR⁴, (CH₂)ₙCH₂NR⁵R⁶, (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl, or aryl;
Hal is F, Cl, Br or I;
SR²⁰ comprises a free or functionally modified thiol group;
R²¹ is H or COSR²¹ forms a pharmaceutically acceptable salt or a pharmaceutically acceptable thioester;
n is 0 or 2;
X is O, S(O)ₚ, NR⁷ or CH₂, with the proviso that X cannot be CH₂ when n is 0;
p is 0, 1 or 2;
NR⁷ comprises a free or functionally modified amino group, e.g., R⁷ is H, alkyl, cycloalkyl, aralkyl, aryl, OH or alkoxy,
A-B, D-E, G-T and J-K are the same or different and are CH₂CH₂, CH═CH or C≡C, with the proviso that at least one of A-B, D-E, G-T and J-K must be CH═CH or C≡C; and
Y is C(O) (i.e., a carbonyl), or Y is 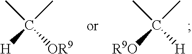
wherein R⁹O constitutes a free or functionally modified hydroxy group;
IX: 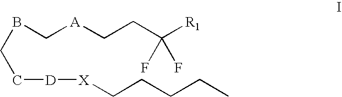
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl, or aryl;
Hal is F, Cl, Br or I;
SR²⁰ comprises a free or functionally modified thiol group;
R²¹ is H or COSR²¹ forms a pharmaceutically acceptable salt or a pharmaceutically acceptable thioester;
A, B, C and D are the same or different and are C₁-C₅ alkyl, alkenyl, or alkynyl or a C₃-C₅ allenyl group;
X is C(O) (i.e. a carbonyl group) or X is 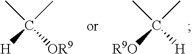
wherein R⁹O constitutes a free or functionally modified hydroxy group;
X: 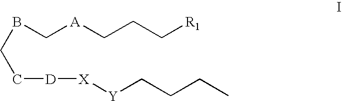
wherein:
R¹ is (CH₂)ₙCO₂R, (CH₂)ₙCONR²R³, (CH₂)ₙCH₂OR⁴, (CH₂)ₙCH₂NR⁵R⁶, (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:
R is H or CO₂R forms a pharmaceutically acceptable salt or a pharmaceutically acceptable ester;
NR²R³ and NR⁵R⁶ are the same or different and comprise a free or functionally modified amino group, e.g., R², R³, R⁵ and R⁶ are the same or different and are H, alkyl, cycloalkyl, aralkyl, aryl, OH, or alkoxy, with the proviso that at most only one of R² and R³ are OH or alkoxy and at most only one of R⁵ and R⁶ are OH or alkoxy;
OR⁴ comprises a free or functionally modified hydroxy group, e.g., R⁴ is H, acyl; alkyl, cycloalkyl, aralkyl, or aryl;
Hal is F, Cl, Br or I;
SR²⁰ comprises a free or functionally modified thiol group;
R²¹ is H or COSR²¹ forms a pharmaceutically acceptable salt or a pharmaceutically acceptable thioester;
n is 0 or 2;
A, B, C and D is C₁-C₅ alkyl, alkenyl, or alkynyl or a C₃-C₅ allenyl group;
Y is 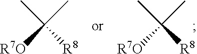
wherein R⁸ is H or CH₃, and
X is CH₂, CH(CH₃) or C(CH₃)₂; or
Y is CH₂, CH(CH₃) or C(CH₃)₂, and X is 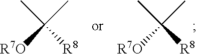
wherein R⁸ is H or CH₃, with the proviso that Y cannot be CH₂ when X is 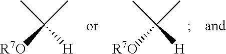
R⁷O comprises a free or functionally modified hydroxy group; and
XI: 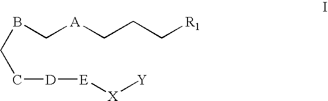
wherein:
R¹ is CO₂R, CONR²R³, CH₂OR₄, CH₂NR⁵R₆, CH₂N₃, CH₂Hal, CH₂NO₂, CH₂SR²⁰, COSR²¹, or 2,3,4,5-tetrazol-1-yl, where:
R is H or a pharmaceutically acceptable cation, or CO₂R forms a pharmaceutically acceptable ester moiety;
NR²R³, NR⁵R⁶ are the same or different and comprise a free or functionally modified amino group;
OR⁴ comprises a free or functionally modified hydroxy group;
Hal is F, Cl, Br, or I;
SR²⁰ comprises a free or functionally modified thiol group;
R²¹ is H or a pharmaceutically acceptable cation, or COSR²¹ forms a pharmaceutically acceptable thioester moiety;
A, B, C, D are the same or different and are C₁-C₅ alkyl, C₂-C₅ alkenyl, C₁₋₅ cyclopropyl, C₂-C₅ alkynyl, or a C₃-C₅ allenyl group;
E is 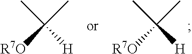
where OR⁷ comprises a free or functionally modified hydroxy group;
X=(CH₂)ₘ or (CH₂)ₘO, where m=1-6; and
Y=a phenyl ring optionally substituted with alkyl, halo, trihalomethyl, acyl, or a free or functionally modified hydroxy, amino, or thiol group; or
X—Y═(CH₂)ₚY¹; where p=0-6; and 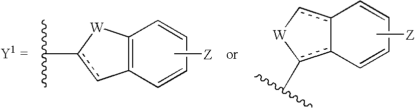
wherein:
W=CH₂, O, S(O)_{q}, NR⁸, CH₂CH₂, CH═CH, CH₂O, CH₂S(O)_{q}, CH═N, or CH₂NR⁸; where q=0-2, and R⁸═H, alkyl, or acyl;
Z=H, alkyl, acyl, halo, trihalomethyl, or a free or functionally modified amino, thiol, or hydroxy group; and
----=single or double bond;
or X—Y=cyclohexyl.

2. The method of claim 1 wherein the HETE compound is a compound of formulas I-III.

3. The method of claim 1 wherein the HETE compound is a compound of formulas I-XI:
I-III: 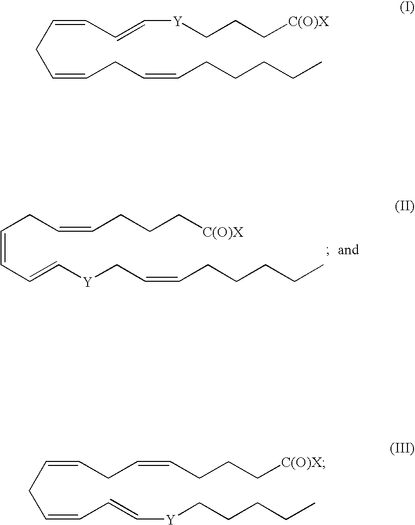
wherein:
X is O⁻M⁺, OR or NHR′;
M⁺ is Na⁺, K⁺, Li⁺, or Cs⁺;
R is H, or substituted or unsubstituted C₁₋₁₅ alkyl, cycloalkyl, (cycloalkyl)alkyl, aryl, or arylalkyl, wherein the substitution is made with a moiety selected from the group consisting of: C₁₋₆ alkyl, fluoro, chloro, bromo, iodo, OH, OCH₂C(O)CH₃, OCH₂C(O)C₂H₅, OCH₃, OCH₂CH₃, OC(O)CH₃, and OC(O)C₂H₅;
R′ is H, or substituted or unsubstituted C₁₋₁₅ alkyl, cycloalkyl, (cycloalkyl)alkyl, aryl, or arylalkyl, wherein the substitution is made with a moiety selected from the group consisting of: C₁₋₆ alkyl, fluoro, chloro, bromo, iodo, OH, OCH₂C(O)CH₃,OCH₂C(O)C₂H₅, OCH₃, OCH₂CH₃, OC(O)CH₃, and OC(O)C₂H₅; and
Y is 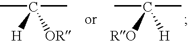
wherein R″ is H or C(O)R;
IV: 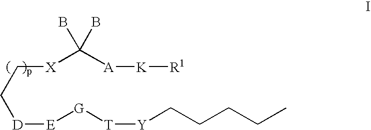
wherein:
R¹ is CO₂R, CONR²R³, CH₂OR⁴, CH₂NR⁵R₆, CH₂N₃, CH₂-Hal, CH₂NO₂, CH₂SR²⁰, COSR²¹, or 2,3,4,5-tetrazol-1-yl, wherein:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R is H or C₁₋₅ alkyl or aryl;
K is C₂-C₈ alkyl, alkenyl, or alkynyl, or a C₃-C₈ allenyl group;
A and X are the same or different and are a direct bond, CH₂, NR⁷, O, or S, with the proviso that at least one of A and X is NR⁷, O, or S;
B is H, or BB together comprises a double bonded O, S, or NR⁸, with the proviso that BB comprises a double bonded O, S, or NR⁸ when A and X are the same or different and are NR⁷, O, or S; wherein:
R⁷ and R⁸ are the same or different and are H, OH, or C₁₋₁₅ alkyl, cycloalkyl, aryl, aralkyl, acyl, or alkoxy;
p is 0 or 1;
D-E, G-H are the same or different and are CH₂CH₂, CH═CH, or C≡C; and
Y is C(O) or 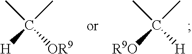
wherein OR⁹ is OH, OCH₂C(O)CH₃,OCH₂C(O)C₂H₅, OCH₃, OCH₂CH₃, OC(O)CH₃, or OC(O)C₂H₅;
V: 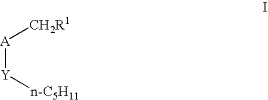
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹, or 2,3,4,5-tetrazol-1-yl, where:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br, or I;
R²⁰ is H or C₁₋₁₅ alkyl or acyl;
R²¹ is H or C₁₋₁₅ alkyl or aryl;
A is L₁-A₁-L₂, L₁-A₂-L₂, L₃-A₂-L₄, or L₅-A₂-L₃;
A₁ is CH₂CH₂;
A₂ is 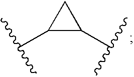
L₁ is CH₂—B-D;
B and D are the same or different and are CH₂CH₂, CH═CH, or C≡C;
L₂ is CH₂—K—CH₂CH₂;
K is CH₂CH₂, CH═CH, or C≡C;
L₃ is CH₂CH₂CH₂, CH₂CH═CH, CH₂C≡C, CH═CHCH₂, C≡CCH₂, or CH═C=CH;
L₄ is X—CH₂CH₂;
X is CH₂CH₂CH═CH, CH₂CH₂C≡C, CH₂CH₂CH₂CH₂, CH₂CH═CHCH₂, CH₂C≡CCH₂, CH═CHCH₂CH₂, C≡CCH₂CH₂, CH₂CH═C═CH, or CH═C=CHCH₂;
L₅ is CH₂CH₂—B-D; and
Y is C(O) or 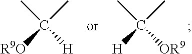
wherein OR⁹ is OH, OCH₂C(O)CH₃,OCH₂C(O)C₂H₅, OCH₃, OCH₂CH₃, OC(O)CH₃, or OC(O)C₂H₅;
VI: 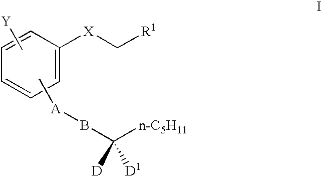
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
X is C₂-C₅ alkyl, alkynyl, or alkenyl or a C₃-C₅ allenyl group;
Y is H, OCH₂C(O)CH₃, OCH₂C(O)C₂H₅, OCH₃, OCH₂CH₃, OC(O)CH₃, or OC(O)C₂H₅, Hal, C(Hal)₃, NH₂, NHCH₃, NHC₂H₅, N(CH₃)₂, NHC(O)CH₃, NHOH, NH(OCH₃), SH, SC(O)CH₃, SCH₃, SC₂H₅, SCH₂C(O)C₂H₅, SCH₂C(O)CH₃, C(O)R, or C₁₋₁₅ alkyl;
R⁷ is H, OH, or C₁₋₁₅ alkyl, alkoxy, amino, alkylamino or alkoxyamino;
A is a direct bond or C₁₋₃ alkyl;
B is CH₂CH₂, cis- or trans-CH═CH, or C≡C; and
one of D and D¹ is H and the other is OH, OCH₂C(O)CH₃, OCH₂C(O)C₂H₅, OCH₃, OCH₂CH₃, OC(O)CH₃, or OC(O)C₂H₅, or DD¹ together comprises a double bonded oxygen;
VII: 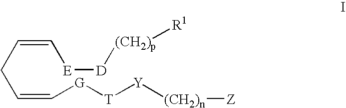
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
E-D is CH₂CH₂CH₂ or cis-CH₂CH═CH; or E is trans-CH═CH and D is CH(X) in either configuration, wherein X is OH, OCH₂C(O)CH₃, OCH₂C(O)C₂H₅, OCH₃, OCH₂CH₃, OC(O)CH₃, or OC(O)C₂H₅; or E is CH₂CH₂ and D is a direct bond;
p is 1 or 3 when E-D is CH₂CH₂CH₂ or cis-CH₂CH═CH, or when E is trans-CH═CH and D is CH(X) in either configuration; or p is 0 when E is CH₂CH₂ and D is a direct bond;
G-T is CH₂CH₂, CH(SR⁷)CH₂ or trans-CH═CH;
R⁷ is H, or C₁₋₁₅ alkyl, aryl, aralkyl, cycloalkyl or acyl; Y is CH(X) in either configuration, or C(O);
n is 0, 2 or 4; and
Z is CH₃, CO₂R, CONR²R³ or CH₂OR⁴;
VIII: 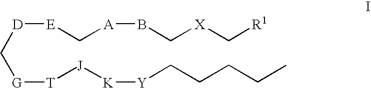
wherein:
R¹ is (CH₂)ₙCO₂R, (CH₂)ₙCONR²R³, (CH₂)ₙCH₂OR⁴, (CH₂)ₙCH₂NR⁵R⁶, (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
n is 0 or 2;
X is O, S(O)ₚ, NR⁷ or CH₂, with the proviso that X cannot be CH₂ when n is 0;
p is 0, 1 or 2;
R⁷ is H, OH or C₁₋₁₅ alkyl, cycloalkyl, aralkyl, aryl, or alkoxy,
A-B, D-E, G-T and J-K are the same or different and are CH₂CH₂, CH═CH or C≡C, with the proviso that at least one of A-B, D-E, G-T and J-K must be CH═CH or C≡C; and
Y is C(O), or 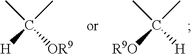
wherein OR⁹ is OH, OCH₂C(O)CH₃,OCH₂C(O)C₂H₅, OCH₃, OCH₂CH₃, OC(O)CH₃, or OC(O)C₂H₅;
IX: 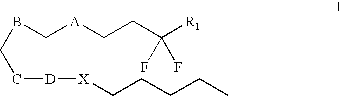
wherein:
R¹ is CO₂R, CONR²R³, CH₂OR⁴, CH₂NR⁵R⁶, CH₂N₃, CH₂Hal, CH₂NO₂, CH₂SR²⁰, COSR²¹ or 2,3,4,5-tetrazol-1-yl, wherein:
R is H, Na⁺, K⁺, Li⁺, Cs⁺, (A)₄N⁺, or C₁₋₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
A, B, C and D are the same or different and are C₁-C₅ alkyl, alkenyl, or alkynyl or a C₃-C₅ allenyl group;
X is C(O) or 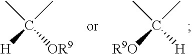
wherein OR⁹ is OH, OCH₂C(O)CH₃,OCH₂C(O)C₂H₅, OCH₃, OCH₂CH₃, OC(O)CH₃, or OC(O)C₂H₅;
X: 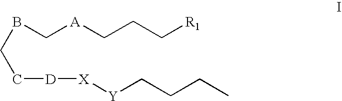
wherein:
R¹ is (CH₂)ₙCO₂R, (CH₂)ₙCONR²R³, (CH₂)ₙCH₂OR⁴, (CH₂)ₙCH₂NR⁵R⁶, (CH₂)ₙCH₂N₃, (CH₂)ₙCH₂Hal, (CH₂)ₙCH₂NO₂, (CH₂)ₙCH₂SR²⁰, (CH₂)ₙCOSR²¹ or (CH₂)ₙ-2,3,4,5-tetrazol-1-yl, wherein:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
n is 0 or 2;
A, B, C and D is C₁-C₅ alkyl, alkenyl, or alkynyl or a C₃-C₅ allenyl group;
Y is 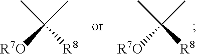
wherein R is H or CH₃, and
X is CH₂, CH(CH₃) or C(CH₃)₂; or
Y is CH₂, CH(CH₃) or C(CH₃)₂, and X is 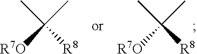
wherein R⁸ is H or CH₃, with the proviso that Y cannot be CH₂ when X is 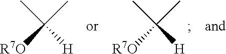
R⁷O is OH, OCH₂C(O)CH₃,OCH₂C(O)C₂H₅, OCH₃, OCH₂CH₃, OC(O)CH₃, or OC(O)C₂H₅; and
XI: 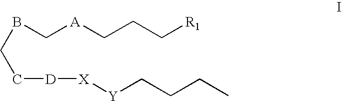
wherein:
R¹ is CO₂R, CONR²R³, CH₂OR₄, CH₂NR⁵R⁶, CH₂N₃, CH₂Hal, CH₂NO₂, CH₂SR²⁰, COSR²¹, or 2,3,4,5-tetrazol-1-yl, where:
R is H, Na⁺, K⁺, Li⁺, Cs⁺, (A)₄N⁺, or C₁₋₁₅ alkyl, cycloalkyl, arylalkyl, aryl or alkoxy;
A is independently H or C₁₋₁₅ alkyl, cycloalkyl, (cycloalkyl)alkyl, alkyl(cycloalkyl), aryl, arylalkyl, heteroaryl, or (A)₄N⁺ forms a heteroaryl, heterocycloalkenyl or heterocycloalkyl ring;
R², R³, R⁵ and R⁶ are the same or different and are H, OH, or C₁₋₁₅ alkyl, cycloalkyl, arylalkyl, aryl, or alkoxy, with the proviso that at most only one of R² and R³ are OH or alkoxy and at most only one of R⁵ and R⁶ are OH or alkoxy;
OR⁴ is OH, OCH₂C(O)CH₃, OCH₂C(O)C₂H₅, OCH₃, OCH₂CH₃, OC(O)CH₃, or OC(O)C₂H₅;
Hal is F, Cl, Br or I;
SR²⁰ is SH, SC(O)CH₃, SCH₃, SC₂H₅, SCH₂C(O)C₂H₅, and SCH₂C(O)CH₃;
R²¹ is H or C₁₋₁₅ alkyl or aryl;
A, B, C, D are the same or different and are C₁-C₅ alkyl, C₂-C₅ alkenyl, C₁₋₅ cyclopropyl, C₂-C₅ alkynyl, or a C₃-C₅ allenyl group;
E is 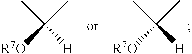
where OR⁷ is OH, OCH₂C(O)CH₃,OCH₂C(O)C₂H₅, OCH₃, OCH₂CH₃, OC(O)CH₃, or OC(O)C₂H₅;
X═(CH₂)ₘ or (CH₂)ₘO, where m=1-6; and
Y=a phenyl ring optionally substituted with C₁₋₆ alkyl or acyl, Hal, C(Hal)₃, OH, OCH₂C(O)CH₃,OCH₂C(O)C₂H₅, OCH₃, OCH₂CH₃, OC(O)CH₃, OC(O)C₂H₅, NH₂, NHCH₃, NHC₂H₅, N(CH₃)₂, NHC(O)CH₃, NHOH, NH(OCH₃)., SH, SC(O)CH₃, SCH₃, SC₂H₅, SCH₂C(O)C₂H₅, or SCH₂C(O)CH₃; and
X—Y═(CH₂)ₚY¹; where p=0-6; and 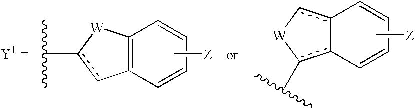
wherein:
W=CH₂, O, S(O)_{q}, NR⁸, CH₂CH₂, CH═CH, CH₂O, CH₂S(O)_{q}, CH═N, or CH₂NR⁸; where q=0-2, and R⁸═H, or C₁₋₁₅ alkyl or acyl;
Z=H, C₁₋₁₅ alkyl or acyl, Hal, C(Hal)₃, OH, OCH₂C(O)CH₃, OCH₂C(O)C₂H₅, OCH₃, OCH₂CH₃, OC(O)CH₃, OC(O)C₂H₅, NH₂, NHCH₃, NHC₂H₅, N(CH₃)₂, NHC(O)CH₃, NHOH, NH(OCH₃)., SH, SC(O)CH₃, SCH₃, SC₂H₅, SCH₂C(O)C₂H₅, or SCH₂C(O)CH₃; and
----=single or double bond;
or X—Y=cyclohexyl.

4. The method of claim 1 wherein the HETE compound is present in the composition in a concentration range of 0.000001 to 10% w/v.

5. The method of claim 4 wherein the HETE compound is present in the composition in a concentration range of 0.00001-0.01% w/v.
